(19) United States
(12) Patent Application Publication
(10) Pub. No.: US 2026/0183044 A1
(43) Pub. Date: Jul. 2, 2026

(54) TECHNOLOGIES FOR CONTROLLING DURATION OF AN ENERGY MODE OF ENERGY-BASED SURGICAL INSTRUMENTS

(71) Applicant: Cilag GmbH International, Zug (CH)

(72) Inventors: Jacob Gee, Cincinnati, OH (US); Tyler Brehm, Cincinnati, OH (US); Scotty Chung, Cincinnati, OH (US); Frederick E. Shelton, IV, Hillsboro, OH (US)

(21) Appl. No.: 19/341,070

(22) Filed: Sep. 26, 2025

Related U.S. Application Data
(60) Provisional application No. 63/740,944, filed on Dec. 31, 2024.

(51) Int. Cl. A61B 18/14; A61B 18/00; A61B 90/00

(52) U.S. Cl. CPC A61B 18/1442; A61B 90/06; A61B 2018/00404; A61B 2018/00601; A61B 2018/0063; A61B 2018/00648; A61B 2018/00666; A61B 2018/00708; A61B 2018/00875; A61B 2018/00994; A61B 2018/1412; A61B 2018/1452; A61B 2090/064

(57) ABSTRACT

A control method for an energy-based surgical instrument includes activating a subtherapeutic radio frequency (RF) signal, activating a stage of an ultrasonic operation, measuring a tissue parameter with the subtherapeutic RF signal, determining whether the tissue parameter exceeds a predetermined threshold, and activating another stage of the ultrasonic operation in response to determining that the tissue parameter exceeds the predetermined threshold. A control method includes measuring a mechanical parameter of the surgical instrument and adjusting delivery of therapeutic energy based on the mechanical parameter and a tissue parameter. Other embodiments are described and claimed.

### CROSS-REFERENCE TO RELATED APPLICATION

This application claims the benefit of and priority to U.S. Patent Application No. 63/740,944, entitled “TECHNOLOGIES FOR THERAPEUTIC AND SUBTHERAPEUTIC CONTROL OF ENERGY-BASED SURGICAL INSTRUMENTS,” which was filed on Dec. 31, 2024, and which is incorporated herein by reference in its entirety.

### TECHNICAL FIELD

The present disclosure relates generally to energy-based surgical instruments and, more particularly, to harmonic and/or electrosurgical surgical instruments.

### BACKGROUND

Energy-based surgical instruments are finding increasingly widespread applications in surgical procedures by virtue of their unique performance characteristics. Depending upon specific device configurations and operational parameters, energy-based surgical instruments can provide both transection of tissue and hemostasis of the tissue by coagulation, which may reduce or otherwise minimize patient trauma. Depending on the particular application, energy-based surgical instruments may utilize different surgical technologies including, for example, ultrasonic and/or electro-surgical (e.g., radio frequency (RF)) technologies.

A typical ultrasonic surgical instrument may include a handpiece containing an ultrasonic transducer and an elongated shaft assembly having a distally mounted end effector to effect the cutting and sealing of tissue. For example, the end effector may include a jaw assembly having an ultrasonic blade and a clamp arm, which may include a non-stick tissue pad or similar bed to receive the ultrasonic blade. In some cases, the elongated shaft assembly may be permanently affixed to the handpiece. In other cases, the elongated shaft assembly may be detachable from the handpiece, as in the case of a disposable shaft assembly or a shaft assembly that is interchangeable between different handpieces. In use, the end effector transmits ultrasonic energy to tissue brought into contact with the ultrasonic blade of the end effector to realize the cutting and sealing action. Such ultrasonic surgical devices may be configured for open surgical use, laparoscopic, and/or endoscopic surgical procedures including robotic-assisted procedures.

Ultrasonic energy cuts and coagulates tissue using temperatures lower than those used in electro-surgical procedures. Vibrating at high frequencies (e.g., 55,500 times per second), the ultrasonic blade denatures protein in the tissue to form a sticky coagulum. Pressure exerted on tissue by the ultrasonic blade surface collapses blood vessels and allows the coagulum to form a hemostatic seal. A surgeon can control the cutting speed and coagulation by the force applied to the tissue by the end effector, the time over which the force is applied, and the selected excursion level of the end effector.

In electro-surgical instruments, one or more electrodes are incorporated into the end effector and configured to apply therapeutic electrical current to the patient's tissue to create a hemostatic seal. In electro-surgical instruments that do not include a harmonic mode (i.e., do not include a harmonic blade), the end effector may be embodied as two clamp arms or jaws. In such embodiments, the electro-surgical instrument may include a separate mechanical knife or blade for cutting the tissue after the creation of the hemostatic seal, which may be incorporated into the elongated shaft attached to the end effector. In bi-polar embodiments, an active electrode may be attached to one of the clamp arms of the end effector and configured to introduce an electrical current into the tissue, which is received by a return electrode attached to the other clamp arm of the end effector (or as the blade itself in embodiments including a harmonic mode). Conversely, in mono-polar embodiments, the return electrode (e.g., a “grounding pad”) may be separate from the electro-surgical instrument and located on a different part of the body of the patient. In some embodiments, the electro-surgical instrument may also be configured to apply a sub-therapeutic electrical current to the patient's tissue, which may be used for sensing purposes (e.g., measuring tissue impedance).

Electro-surgery forms hemostatic seals by generating heat in the tissue via the introduced electrical energy, which is embodied as radio frequency (“RF”) energy. The particular frequency employed can vary based on the intended use of the electro-surgical instrument within the range of about 100 kHz to 1 MHz, although higher frequencies can be employed in some embodiments. Additionally, sub-therapeutic frequencies may be used in some situations for purposes other than hemostatic sealing, such as performing various electrical measurements on the tissue.

It should be appreciated that some energy-based surgical instruments may employ dual or multi-modal technologies for the transection and/or hemostasis of patient tissue. For example, in some cases, an energy-based surgical instrument may include both ultrasonic and electro-surgical capabilities (e.g., by utilizing the ultrasonic blade as an electrode for the electro-surgery mode), which increases the surgical options provided by the surgical instrument to the surgeon.

### SUMMARY

According to an aspect of the present disclosure, a method for controlling a surgical instrument includes applying, by a control element, a subtherapeutic radio frequency (RF) signal to tissue of a patient with an electrode of an end effector of the surgical instrument; activating, by the control element, a first stage of an ultrasonic operation, wherein the first stage applies ultrasonic energy to an ultrasonic blade of the end effector of the surgical instrument; measuring, by the control element, a tissue parameter with the subtherapeutic RF signal while activating the first stage of the ultrasonic operation; determining, by the control element, whether the tissue parameter has a predetermined relationship to a predetermined threshold; and activating, by the control element, a second stage of the ultrasonic operation in response to determining that the tissue parameter has the predetermined relationship to the predetermined threshold.

In some embodiments, the tissue parameter comprises tissue impedance. In some embodiments, the tissue parameter comprises change in tissue impedance.

In some embodiments, the first stage comprises a pre-heating stage and the second stage comprises a vessel sealing stage. In some embodiments, activating the second stage of the ultrasonic operation includes applying ultrasonic energy to the ultrasonic blade at an energy level based on the tissue parameter.

In some embodiments, the method further includes measuring, by the control element, the tissue parameter with the subtherapeutic RF signal while activating the second stage of the ultrasonic operation; determining, by the control element, whether the tissue parameter has a predetermined relationship to a second predetermined threshold; and activating, by the control element, a third stage of the ultrasonic operation in response to determining that the tissue parameter has the predetermined relationship to the second predetermined threshold. In some embodiments, the first stage comprises a pre-heating stage, the second stage comprises a vessel sealing stage, and the third stage comprises a transection stage. In some embodiments, activating the first stage of the ultrasonic operation includes applying ultrasonic energy at a first energy level; activating the second stage of the ultrasonic operation includes applying ultrasonic energy at a second energy level lower than the first energy level; and activating the third stage of the ultrasonic operation includes applying ultrasonic energy at a third energy level higher than the second energy level. In some embodiments, the method further includes measuring, by the control element, the tissue parameter with the subtherapeutic RF signal while activating the third stage of the ultrasonic operation; determining, by the control element, whether the tissue parameter has a predetermined relationship to a third predetermined threshold; and deactivating, by the control element, the ultrasonic operation in response to determining that the tissue parameter has the predetermined relationship to the third predetermined threshold.

According to another aspect, a method for controlling a surgical instrument includes measuring, by a control element, a first mechanical parameter of an end effector of the surgical instrument; sensing, by the control element, a first tissue parameter while applying energy to tissue of the patient, wherein the energy comprises radio frequency (RF) energy or ultrasonic energy; and adjusting, by the control element, delivery of therapeutic energy with the surgical energy based on the first mechanical parameter and the first tissue parameter.

In some embodiments, the first mechanical parameter comprises angle of jaw opening. In some embodiments, the first mechanical parameter comprises angle of initial tissue contact or time of initial tissue contact. In some embodiments, the first mechanical parameter comprises force on the jaws.

In some embodiments, the first tissue parameter comprises tissue impedance. In some embodiments, the first tissue parameter comprises transducer impedance. In some embodiments, the first tissue parameter comprises impedance over time. In some embodiments, the first tissue parameter comprises spectroscopy indicative of tissue composition.

In some embodiments, adjusting the delivery of the therapeutic energy includes determining a tissue type. In some embodiments, adjusting the delivery of the therapeutic energy includes determining a stage of a surgical operation. In some embodiments, adjusting the delivery of the therapeutic energy includes selecting an energy modality of the therapeutic energy, wherein the energy modality is selected from RF energy or ultrasonic energy. In some embodiments, adjusting the delivery of the therapeutic energy includes changing the energy modality of the therapeutic energy. In some embodiments, adjusting the delivery of the therapeutic energy includes determining a time for changing the energy modality. In some embodiments, adjusting the delivery of the therapeutic energy includes determining a power level of the therapeutic energy. In some embodiments, adjusting the delivery of the therapeutic energy includes starting or stopping the delivery of the therapeutic energy.

According to another aspect, a system for controlling a surgical instrument includes the surgical instrument and a control element. The surgical instrument includes an end effector having an electrode configured to apply radio frequency (RF) energy to tissue of a patient, and an ultrasonic blade configured to apply ultrasonic energy to the tissue of the patient. The control element is configured to apply a subtherapeutic RF signal the to tissue of the patient with the electrode of the end effector, activate a first stage of an ultrasonic operation, wherein the first stage applies ultrasonic energy to the ultrasonic blade of the end effector, measure a tissue parameter with the subtherapeutic RF signal during activation of the first stage of the ultrasonic operation, determine whether the tissue parameter has a predetermined relationship to a predetermined threshold, and activate a second stage of the ultrasonic operation in response to a determination that the tissue parameter has the predetermined relationship to the predetermined threshold.

In some embodiments, the tissue parameter comprises tissue impedance. In some embodiments, the tissue parameter comprises change in tissue impedance.

In some embodiments, the first stage comprises a pre-heating stage and the second stage comprises a vessel sealing stage. In some embodiments, to activate the second stage of the ultrasonic operation comprises to apply ultrasonic energy to the ultrasonic blade at an energy level based on the tissue parameter.

In some embodiments, the control element is further configured to measure the tissue parameter with the subtherapeutic RF signal during activation of the second stage of the ultrasonic operation; determine whether the tissue parameter has a predetermined relationship to a second predetermined threshold; and activate a third stage of the ultrasonic operation in response to a determination that the tissue parameter has the predetermined relationship to the second predetermined threshold. In some embodiments, the first stage comprises a pre-heating stage, the second stage comprises a vessel sealing stage, and the third stage comprises a transection stage. In some embodiments, to activate the first stage of the ultrasonic operation includes to apply ultrasonic energy at a first energy level; to activate the second stage of the ultrasonic operation includes to apply ultrasonic energy at a second energy level lower than the first energy level; and to activate the third stage of the ultrasonic operation includes to apply ultrasonic energy at a third energy level higher than the second energy level. In some embodiments, the control element is further configured to measure the tissue parameter with the subtherapeutic RF signal during activation of the third stage of the ultrasonic operation; determine whether the tissue parameter has a predetermined relationship to a third predetermined threshold; and deactivate the ultrasonic operation in response to a determination that the tissue parameter has the predetermined relationship to the third predetermined threshold.

According to another aspect, a system for controlling a surgical instrument includes the surgical instrument and a control element. The surgical instrument includes an end effector having an electrode configured to apply radio frequency (RF) energy to tissue of a patient, and an ultrasonic blade configured to apply ultrasonic energy to the tissue of the patient. The control element is configured to measure a first mechanical parameter of the end effector, sense a first tissue parameter during application of energy to tissue of the patient, wherein the energy comprises radio frequency (RF) energy or ultrasonic energy, and adjust delivery of therapeutic energy with the surgical energy based on the first mechanical parameter and the first tissue parameter.

In some embodiments, the first mechanical parameter comprises angle of jaw opening. In some embodiments, first mechanical parameter comprises angle of initial tissue contact or time of initial tissue contact. In some embodiments, the first mechanical parameter comprises force on the jaws.

In some embodiments, the first tissue parameter comprises tissue impedance. In some embodiments, the first tissue parameter comprises transducer impedance. In some embodiments, the first tissue parameter comprises impedance over time. In some embodiments, the first tissue parameter comprises spectroscopy indicative of tissue composition.

In some embodiments, to adjust the delivery of the therapeutic energy includes to determine a tissue type. In some embodiments, to adjust the delivery of the therapeutic energy includes to determine a stage of a surgical operation. In some embodiments, to adjust the delivery of the therapeutic energy includes to select an energy modality of the therapeutic energy, wherein the energy modality is selected from RF energy or ultrasonic energy. In some embodiments, to adjust the delivery of the therapeutic energy includes to change the energy modality of the therapeutic energy. In some embodiments, to adjust the delivery of the therapeutic energy includes to determine a time for changing the energy modality. In some embodiments, to adjust the delivery of the therapeutic energy includes to determine a power level of the therapeutic energy. In some embodiments, to adjust the delivery of the therapeutic energy includes to start or to stop the delivery of the therapeutic energy.

### BRIEF DESCRIPTION OF THE DRAWINGS

The detailed description particularly refers to the following figures, in which:

FIG. 1 is a simplified diagram of an embodiment of a system for performing an energy-based surgical procedure;

FIG. 2 is a perspective view of an embodiment of an energy-based surgical instrument of the system of FIG. 1;

FIG. 3 is a side elevation view of a jaw assembly of an end effector of the surgical instrument of FIG. 2 including an ultrasonic blade and in an open state;

FIG. 4 is a side elevation view of the jaw assembly of the end effector of the surgical instrument of FIG. 2 including an ultrasonic blade and in a closed state;

FIG. 5A is a perspective view of another embodiment of the end effector of the surgical instrument of FIG. 2 including an electrode on a lower jaw clamp of the jaw assembly;

FIG. 5B is a perspective view of another embodiment of the end effector of the surgical instrument of FIG. 2 including two jaw clamps, each having an electrode attached thereto;

FIG. 6 is an exploded view of the surgical instrument of FIG. 2;

FIG. 7 is a block diagram of a control circuit of the surgical instrument of FIG. 2;

FIG. 8 is a simplified flow diagram of at least one method for controlling an energy-based surgical instrument;

FIG. 9 is a simplified flow diagram of a method for controlling a combined energy-based surgical instrument;

FIG. 10 is a chart illustrating operation of the method of FIG. 9; and

FIG. 11 is a simplified flow diagram of a method for controlling a combined energy-based surgical instrument according to mechanical parameters of the instrument.

### DETAILED DESCRIPTION OF THE DRAWINGS

While the concepts of the present disclosure are susceptible to various modifications and alternative forms, specific illustrative embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, distal, proximal, et cetera, may be used throughout the specification in reference to the surgical instruments described herein as well as in reference to the patient's natural anatomy. Such terms have well-understood meanings in both the study of anatomy and the field of surgery. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

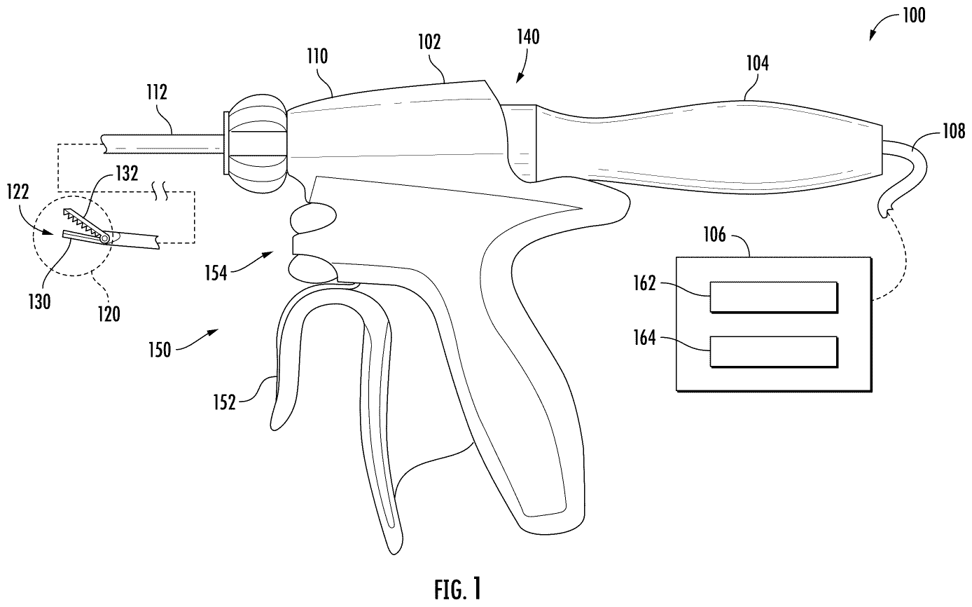

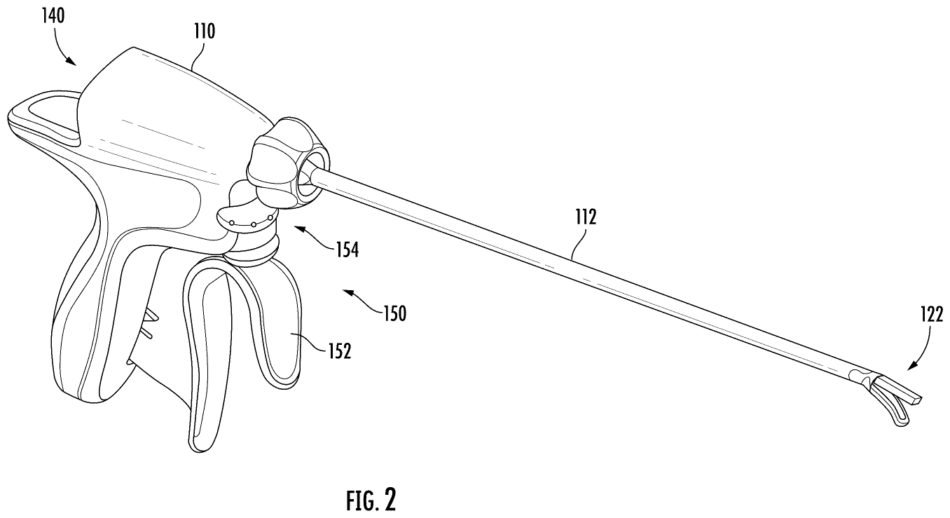

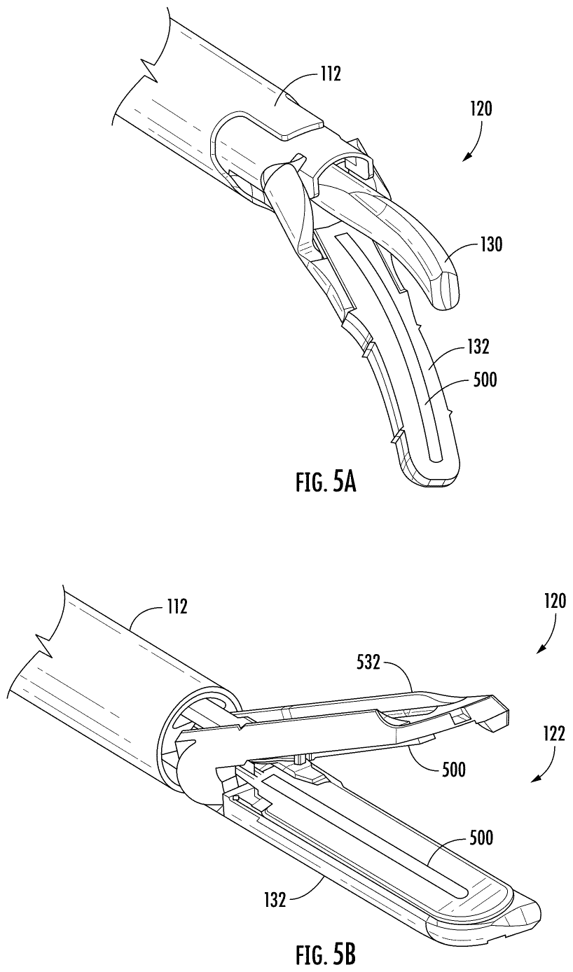

Referring now to FIGS. 1 and 2, in an illustrative embodiment, a system 100 for performing an energy-based surgical procedure includes a surgical instrument 102, a transducer 104, and a generator 106. The surgical instrument 102 is illustratively embodied as an ultrasonic surgical instrument, but may be embodied as an electro-surgical surgical instrument or a multi-modal, ultrasonic/elector-surgical surgical instrument in other embodiments. In use, the surgical instrument 102 is usable to perform various surgical procedures including laparoscopic, endoscopic, or traditional open surgical procedures. In doing so, a surgeon may selectively activate an ultrasonic mode (and/or an electro-surgical/RF mode) of the surgical instrument 102. In the ultrasonic mode, the generator 106 drives the transducer 104 to cause an ultrasonic blade 130 of a jaw assembly 122 of an end effector 120 of the surgical instrument 102 to vibrate at a reference frequency, which facilitates the contemporaneous cutting and hemostatic sealing of patient tissue. Additionally or alternatively, in some embodiments, the surgeon may selectively activate an electro-surgical mode of the surgical instrument 102 to deliver an amount of therapeutic RF energy to the patient tissue to effect hemostatic sealing. In such embodiments, the blade 130 may be embodied as an ultrasonic blade 130 or as a mechanical blade designed to cut tissue using mechanical force (e.g., in those embodiments not employing ultrasonic technologies). Furthermore, in some embodiments, the surgical instrument 102 may be configured with only an electro-surgical/RF mode and, in such embodiments, the jaw assembly 122 of the end effector 120 may not include the ultrasonic blade 130 as discussed in more detail below in regard to FIG. 5B.

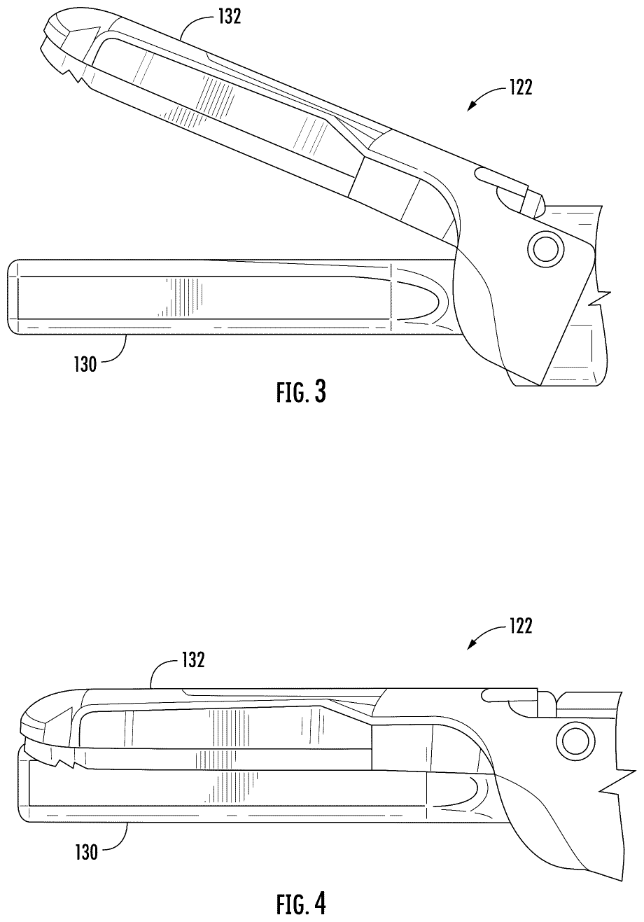

The surgical instrument 102 is illustratively embodied as ultrasonic surgical shears but may be embodied as other types of surgical instruments having an ultrasonic mode and/or electro-surgical mode in other embodiments. In the illustrative embodiment, the surgical instrument 102 includes a handle assembly 110 and an elongated shaft assembly 112, which extends distally away from the handle assembly 110 and may be removably attached to the handle assembly 110 in some embodiments. The elongated shaft assembly 112 includes the end effector 120 located at a distal end opposite the handle assembly 110. The end effector 120 includes the jaw assembly 122, which illustratively includes the ultrasonic blade 130 and a corresponding jaw clamp 132 (but may include two jaw clamps in those embodiments having only an electro-surgical/RF mode). As shown in FIGS. 3 and 4, the jaw assembly 122 is movable between an open state (FIG. 3) in which the jaw clamp 132 is positioned away from the ultrasonic blade 130 and a closed state (FIG. 4) in which the jaw clamp 132 is positioned near or otherwise contacts the ultrasonic blade 130. Actuation of the jaw assembly 122 from the open state to the closed state allows for the grasping, cutting, and coagulation of vessels and/or tissue by the jaw assembly 122. It should be appreciated that the open state may correspond to a degree of openness that is less than a fully opened position of the jaw assembly 122 and the closed state may correspond to a degree of closeness that is less than a fully closed position. That is, the closed state may, for example correspond to a minimal distance between the distal ends of the jaw clamp 132 and the ultrasonic blade 130 and the open state may correspond to a maximum distance between the distal ends of the jaw clamp 132 and the ultrasonic blade 130. However, in other embodiments, the open state may correspond to a fully opened position of the jaw assembly 122 and the closed state may correspond to a fully closed position of the jaw assembly 122.

In those embodiments in which the surgical instrument 102 includes both a ultrasonic mode and an electro-surgical/RF mode, the end effector 120 may include one or more RF electrodes 500 incorporated into the jaw clamp 132 as shown in FIG. 5A. Although the illustrative end effector 120 includes only a single electrode 500 in the embodiment of FIG. 5A, it should be appreciated that the end effector 120 may include additional electrodes 500 in other embodiments (e.g., multiple pads of electrodes 500). The electrode(s) 500 may be embodied as an active electrode configured to the RF energy or as a return electrode configured to “sink” an applied RF energy. In those embodiments utilizing bi-polar RF implementation, the ultrasonic blade 130 may embody the active or return electrode, with the electrode 500 embodying the other active or return electrode. Alternatively, other active or return electrodes may be incorporated on the ultrasonic blade 130 or in another part of the jaw assembly 122 of the end effector 120. In mono-polar implementation, the RF electrode(s) 500 may be embodied as an active electrode, and a return electrode may be attached to a portion of the patient's body.

In those embodiments in which the surgical instrument 102 includes only an electro-surgical/RF mode, the jaw assembly 122 of the end effector 120 includes a jaw clamp 532 in place of the ultrasonic blade 130 as shown in FIG. 5B. In such embodiments, an electrode 500 may be attached to or otherwise incorporated into each jaw clamp 132, 532 and be embodied as an active or a return electrode to facilitate the application of RF energy to tissue captured between the jaw clamps 132, 532. In such embodiments, the surgical instrument 102 may include a knife incorporated into the elongated shaft assembly 112 that is configured to eject outwardly to cut the patient's tissue after sealing of the tissue by the RF energy.

Referring back to FIGS. 1 and 2, in those embodiments including ultrasonic capabilities, the handle assembly 110 includes a receptacle 140 configured to receive the transducer 104 to facilitate connection of the transducer 104 to the handle assembly 110 and the elongated shaft assembly 112. The handle assembly 110 also includes a trigger assembly 150, which includes a primary trigger 152 and a switch assembly 154. The primary trigger 152 is operable by the surgeon to move the jaw assembly 122 of the end effector 120 between the open and closed states. The switch assembly 154 includes one or more buttons, which are selectable by the surgeon to activate (and configure, in some embodiments) the ultrasonic mode and/or the electro-surgical mode of the surgical instrument 102.

The transducer 104 is illustratively connected to the generator 106 by a cable assembly 108. As discussed above, the generator 106 is configured to drive the transducer 104 at a reference or resonant frequency to thereby cause the ultrasonic blade 130 to vibrate. For example, in an illustrative embodiment, the generator 106 may supply an electrical signal to the transducer 104 to cause the ultrasonic blade 130 of the jaw assembly 122 to vibrate longitudinally in the range of, for example, approximately 20 kHz to 250 kHz. In particular embodiments, for example, the ultrasonic blade 130 may vibrate in the range of about 54 kHz to 56 kHz (e.g., at about 55.5 kHz). In other embodiments, the ultrasonic blade 130 may vibrate at other frequencies including, for example, about 31 kHz or about 80 kHz. The excursion of the vibrations at the ultrasonic blade 130 can be controlled by, for example, controlling the amplitude of the electrical signal applied to the transducer 104 by the generator 106. The generator 106 may be activated so that electrical energy may be continuously or intermittently supplied to the transducer 104. The generator 106 also has a power line (not shown) for insertion in an electro-surgical unit or conventional electrical outlet. Additionally or alternatively, the generator 106 may be powered by a direct current (DC) source, such as a battery.

In some embodiments, the generator 106 may be configured to operate in different modes. In such embodiments, the generator 106 may include an ultrasonic generator module 162 for controlling an ultrasonic mode, an electro-surgical/Radio Frequency (RF) generator module 164 for controlling an electro-surgical mode, and/or other generator modules (e.g., a heat generator module) for controlling other operation modes. The various modes of the generator 106 may be operated independently of each other in some embodiments. For example, the generator 106 may activate the ultrasonic mode of the ultrasonic generator module 162 to apply ultrasonic energy to the jaw assembly 122 and subsequently, either therapeutic or sub-therapeutic RF energy may be applied to the jaw assembly 122 by the electro-surgical generator module 164. Alternatively, the activation modes of the generator 106 may be operated simultaneously or contemporaneously with each other.

In the electro-surgical mode, the electro-surgical generator module 164 is configured to generate RF energy at a frequency in the range of about 100 kilohertz (100 kHz) to about 1 megahertz (1 MHz). The generated RF energy is supplied to the patient's tissue via the electrodes 500 of the end effector 120 as described above in regard to FIG. 5. In some embodiments, the electro-surgical generator module 164 may also be configured to selectively provide the RF energy at sub-therapeutic levels to perform various electrical measurements of the patient's tissue. For example, the electro-surgical generator module 164 may be configured to measure an impedance of the patient's tissue using the electrodes 500 and a suitable RF energy level.

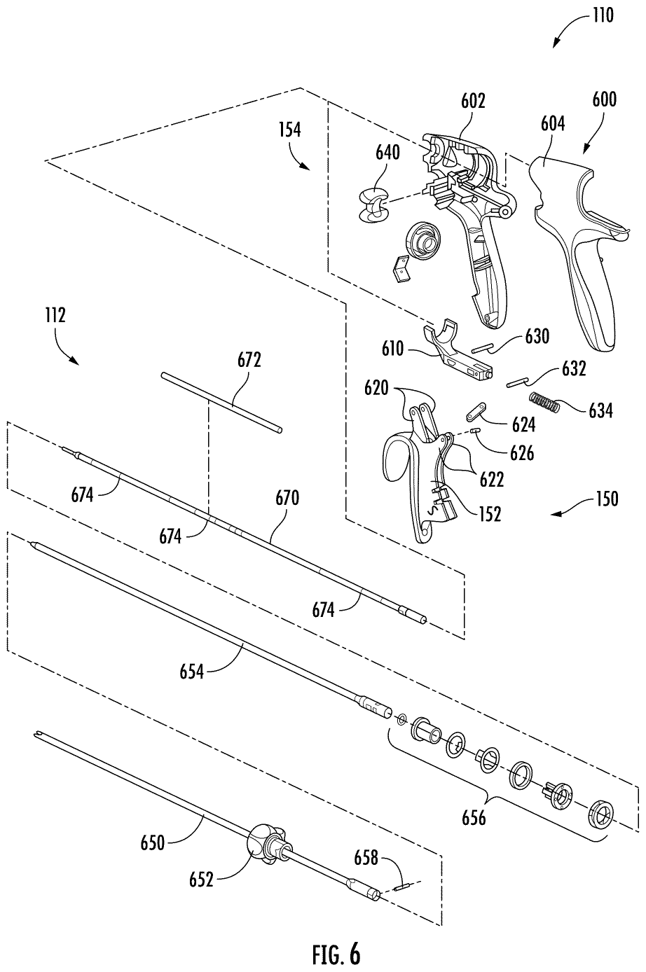

Referring now to FIG. 6, as discussed above, the illustrative surgical instrument 102 includes the handle assembly 110 and the elongated shaft assembly 112, which extends distally away from the handle assembly 110. The handle assembly 110 includes a housing 600, which includes a right half-housing 602 and a left half-housing 604. The half-housings 602, 604 are configured to mate with each other to form the housing 600. To facilitate such mating, each of the half-housings 602, 604 may include various interfaces sized to mechanically align and engage one another to form the housing 600 and enclose the internal working components of the surgical instrument 102.

The primary trigger 152 of the trigger assembly 150 is coupled to a linkage mechanism to translate the rotational motion of the primary trigger 152 to axial motion of a yoke 610, which in turn is configured to move the jaw assembly 122 of the end effector 120 between the open and closed states via the elongated shaft assembly 112. The primary trigger 152 includes a first set of flanges 620 having openings formed therein to receive a first yoke pin 630, which extends through the yoke 610. The primary trigger 152 also includes a second set of flanges 622 configured to receive a first end of a link 624. A trigger pin 626 is received in openings formed in the first end of the link 624 and the second set of flanges 622. The trigger pin 626 forms a trigger pivot point for the primary trigger 152. A second end of the link 624, opposite the first end, is received in a slot formed in a proximal end of the yoke 610 and retained therein by a second yoke pin 632. As the primary trigger 152 is rotated about the pivot point formed from the trigger pin 626, the yoke 610 translates horizontally. A spring 634 is used to bias the yoke forward such that the jaw assembly 122 of the end effector 120 is biased to the open state (or a fully opened state).

As discussed above, the trigger assembly 150 also includes a switch assembly 154. The switch assembly 154 illustratively includes a toggle switch 640, which is selectable to activate one or more switches 642. Activation of the switches 642 electrically energizes an electrical element 644, which electrically energizes the ultrasonic transducer 104 to engage the ultrasonic mode of the surgical instrument 102.

The elongated shaft assembly 112 includes an outer tubular sheath 650 and a rotation knob 652 coupled to the outer cylindrical sheath 650. The rotation knob 652 is operable to rotate the outer cylindrical sheath 650 about an axis defined by the outer cylindrical sheath 650. A reciprocating tubular actuator 654 is located within the outer tubular sheath 650 and mechanically engaged with the end effector 120 on a distal end. The reciprocating tubular actuator 654 is also mechanically engaged, on a proximal end, with the yoke 610 within the handle assembly 110 via coupling elements 656. In embodiments including an ultrasonic mode, an ultrasonic waveguide 670 is located within the reciprocating tubular actuator 654. A distal end of the ultrasonic waveguide 670 is acoustically coupled (e.g., directly or indirectly mechanically coupled) to the ultrasonic blade 130, and a proximal end is acoustically coupled to the transducer 104. The ultrasonic waveguide 670 may be isolated from other components of the elongated shaft assembly 112 by a protective sheath 672 and a number of isolation elements 674. The outer tubular sheath 650, the reciprocating tubular actuator 654, and the ultrasonic waveguide 670 are mechanically engaged together via a pin 658.

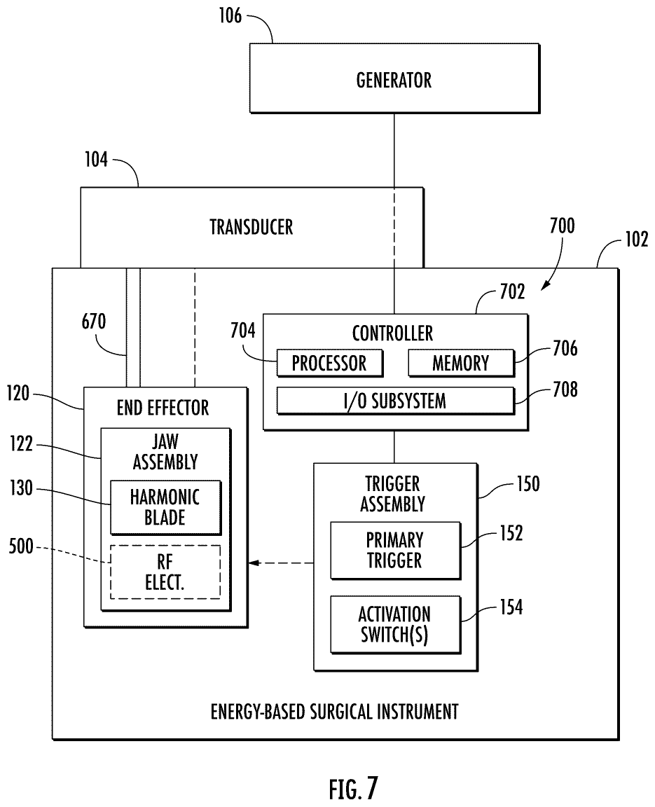

Referring now to FIG. 7, in the illustrative embodiment, the surgical instrument 102 includes a control circuit 700. The control circuit 700 includes a controller 702 and the trigger assembly 150, which cooperate to provide ultrasonic energy to the harmonic blade 130 of the jaw assembly 122 of the end effector 120 and/or RF energy to the RF electrodes 500 of the jaw assembly 122, depending on the operation modes of the surgical instrument 102 as discussed above. In other embodiments, however, the control circuit 700 may include additional or other electronic devices and/or circuit.

The controller 702 may be embodied as any type of controller, functional block, digital logic, or other component, device, circuitry, or collection thereof capable of performing the functions described herein. In illustrative embodiment, the controller 702 includes a processor 704, a memory 706, and an input/output (I/O) subsystem 708. The processor 704 may be embodied as any type of processor capable of performing the functions described herein. For example, the processor 704 may be embodied as a single or multi-core processor(s), digital signal processor, microcontroller, or other processor or processing/controlling circuit. Similarly, the memory 706 may be embodied as any type of volatile and/or non-volatile memory or data storage capable of performing the functions described herein. In operation, the memory 706 may store various data and software used during operation of the control circuit 700 such as executable firmware or software, programs, libraries, and drivers, which may be executed or otherwise used by the processor 704.

The processor 704 and memory 706 are communicatively coupled to other components of the control circuit 700 via the I/O subsystem 708, which may be embodied as circuitry and/or components to facilitate input/output operations between the controller 702 (e.g., the processor 704 and the memory 706) and the other components of the control circuit 700. For example, the I/O subsystem 708 may be embodied as, or otherwise include, memory controller hubs, input/output control hubs, firmware devices, communication links (i.e., point-to-point links, bus links, wires, cables, light guides, printed circuit board traces, etc.) and/or other components and subsystems to facilitate the input/output operations. In some embodiments, the I/O subsystem 708 may form a portion of a system-on-a-chip (SoC) and be incorporated, along with the processor 704 and the memory 706, and other components of the surgical instrument 102, on a single integrated circuit chip. Additionally, in some embodiments, the memory 706, or portions of the memory 706, may be incorporated into the processor 704.

During operation, as discussed above, the controller 702 is configured to control activation of an ultrasonic mode and/or an electro-surgical/RF mode of the surgical instrument 102. To do so, the controller 702 may monitor for activation of the primary trigger 152 and/or one or more activation switches 154 of the trigger assembly 150. In response to activation of the appropriate trigger 152 or switch 154, the controller 702 controls the transducer 104 to generate the ultrasonic energy, which is propagated to the harmonic blade 130 via the ultrasonic waveguide 670. Additionally or alternatively, in response to activation of a corresponding switch 154 of the trigger assembly 150, the controller 702 may be configured to supply an amount of RF energy, via the electro-surgical generator module 164 to the RF electrodes 500 via interconnections 710. It should be appreciated that, although the transducer 104 and the generator 106 are shown as separate components from the energy-based surgical instrument 102 in FIGS. 1 and 7, the transducer 104 and/or the generator 106 may be incorporated into the surgical instrument 102 in other embodiments.

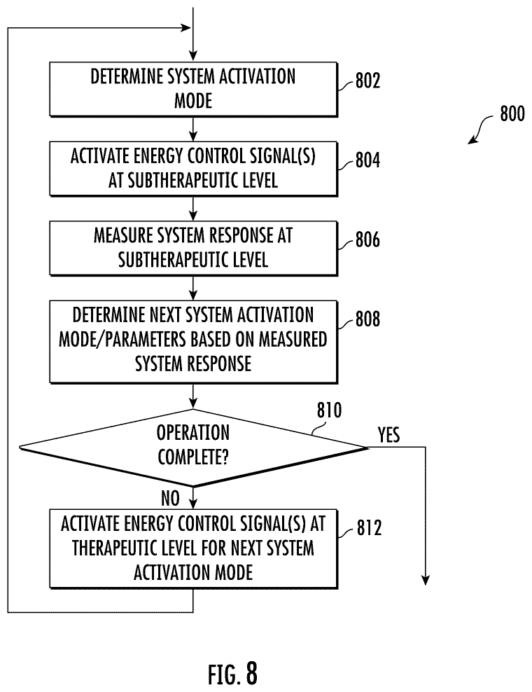

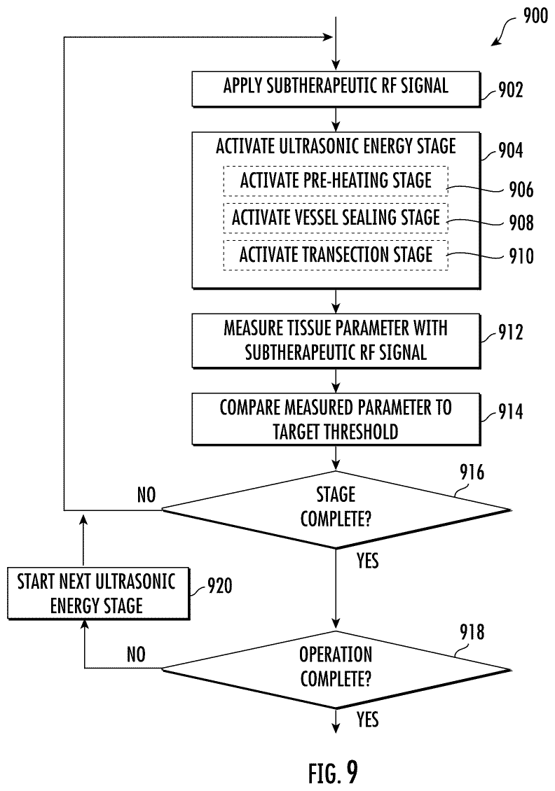

Referring now to FIG. 8, a method 800 for controlling an energy-based surgical instrument 102 is shown. The method 800 may be executed by the controller 702, the generator 106, and/or one or more other microcontrollers or other control elements of the system 100. The method 800 begins in block 802, in which the control element determines a system activation mode for the surgical instrument 102. The system activation mode may include an energy modality (e.g., RF, ultrasound, combined RF and ultrasound, etc.), a surgical operation or firing to be performed with the surgical instrument 102 (e.g., seal, transect, seal and transect, etc.), and/or a sub-operation or phase (e.g., heating, sensing, sealing, cutting, etc.).

In block 804, the control element activates one or more energy control signals at a subtherapeutic level. The subtherapeutic level may be a lower power or energy level that does not cause coagulation, transection, or other therapeutic actions in tissue. The subtherapeutic level may cause other responses in the tissue, such as subtherapeutic heating. The subtherapeutic control may activate ultrasound energy, RF energy, or combined ultrasound and RF energy at the subtherapeutic level.

In block 806, the control element measures a system response at the subtherapeutic level. For example, the control element may measure tissue impedance, acoustic impedance, frequency shift, phase shift, or other responses to application of the subtherapeutic signal.

In block 808, the control element determines a next system activation mode and/or parameters based on the measured system response. For example, the control element may determine whether to switch energy modalities (e.g., from RF to ultrasound, from ultrasound to RF, from a single modality to a combined modality, or other change in energy modality). As another example, the control element may determine whether to change sub-operation or phase, e.g., from pre-heating to sealing, from sealing to transecting, or other change in sub-operation. As another example, the control element may determine one or more parameters for application of therapeutic levels of energy, such as setpoint, amplitude, frequency, crest factor (CF), or other parameters. As yet another example, the control element may determine that the surgical operation (e.g., sealing and/or transecting tissue) has been completed.

In block 810, the control element checks whether the present surgical operation or firing has been completed. If so, the method 800 is completed. The method 800 may be executed again in response to subsequent surgical firings. If the surgical operation is not complete, the method 800 advances to block 812.

In block 812, the control element activates one or more energy control signals at a therapeutic level for the next system activation mode determined as described above. For example, the control element may activate ultrasound and/or RF energy at a setpoint determined as described above or otherwise cause activation of the surgical instrument 102. After activation, the method 800 may loop back to block 802 to continue performing subtherapeutic measurement and control of therapeutic energy application.

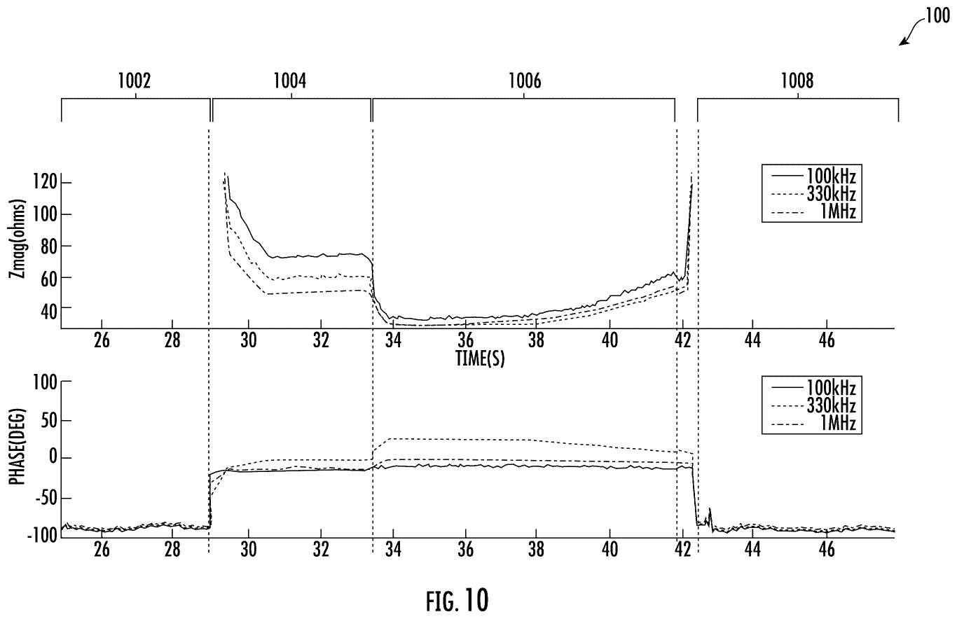

Additionally or alternatively, in some embodiments the control element may perform the operations of the method 800 in a different order and/or in a different combination. Further, in some embodiments the control element may perform additional or different operations and/or make additional or different measurements. Illustrative examples of control operations that may be performed in connection with the surgical instrument 102 are described further below in connection with FIGS. 9-11.

Referring now to FIG. 9, a method 900 for controlling an energy-based surgical instrument 102 is shown. The method 900 may be executed by the controller 702, the generator 106, and/or one or more other microcontrollers or other control elements of the system 100. The method 900 may be executed, for example, in connection with combined RF and ultrasound operation of the surgical instrument 102. The method 900 begins in block 902, in which the control element applies a subtherapeutic RF signal. The subtherapeutic RF signal includes RF energy applied using the electrode 500 at a subtherapeutic level. That is, the applied subtherapeutic RF signal does not cause tissue sealing and/or transection.

In block 904, the control element activates an ultrasonic energy stage, in which the ultrasonic blade 130 applies ultrasonic energy to the tissue. The ultrasonic energy stage is a part of a multi-stage ultrasonic operation, such as a seal and transect operation. The level of energy applied during the ultrasonic energy stage depends on the operation performed during the current stage. In some embodiments, in block 906 the control element activates a pre-heating stage. The pre-heating stage applies ultrasonic energy to the ultrasonic blade 130 (and thus the surrounding tissue) until the blade 130 and/or tissue reaches a target temperature. In some embodiments, in block 908 the control element activates a vessel sealing stage. The vessel sealing stage applies ultrasonic energy to heat and coagulate tissue. The applied energy level (i.e., tip displacement, transducer current, or other power level) in the vessel sealing stage may be lower than the energy level applied during the pre-heating stage. In some embodiments, in block 910 the control element activates a transection stage. The transection stage, applied after the blood vessel is completely sealed, cuts or otherwise transects the blood vessel. The applied energy level in the transection stage may be greater than the energy level applied during the vessel sealing stage.

In block 912, the control element senses a tissue parameter based on the subtherapeutic RF signal. The control element may measure tissue impedance, rate of change of impedance, or other tissue parameter that may be measured with the subtherapeutic RF sensing signal. The control element senses the tissue parameter during application of the ultrasonic energy. Accordingly, the control element may monitor changes in the tissue parameter (e.g., changes in tissue impedance).

In block 914, the control element compares the measured tissue parameter to a predetermined target parameter. For example, the control element may compare measured tissue impedance to a target impedance. If the measured impedance exceeds the target impedance, then the current ultrasonic energy stage may be completed. As another example, the measured impedance exceeds a target rate of change of impedance, then the current ultrasonic energy stage may be completed. In other embodiments, the control element may compare the tissue parameter to the target parameter to determine whether any predetermined relationship exists between those parameters (e.g., greater than, less than, equal to, greater than or equal to, less than or equal to, etc.).

In block 916, the control element determines whether the current ultrasonic energy stage is complete based on the comparison of the measured tissue parameter to the target threshold. If the stage is not complete, the method 900 loops back to block 904. If the stage is complete, the method 900 advances to block 918.

In block 918, the control element determines whether the entire multi-stage ultrasonic operation is complete. For example, the control element may determine whether all of the pre-heating, vessel sealing, and transection stages have completed. If so, the method 900 is completed. If not, the method 900 branches to block 920, in which the control element starts the next ultrasonic energy stage and then loops back to block 904 to continue applying ultrasonic energy. For example, the control element may advance from the pre-heating stage to the vessel sealing stage, or the control element may advance from the vessel sealing stage to the transection stage, as appropriate.

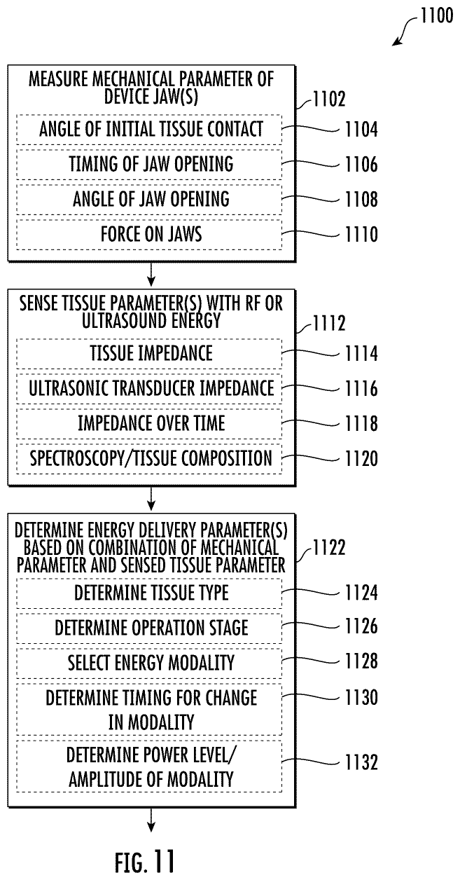

Referring now to FIG. 10, diagram 1000 illustrates tissue parameter measurements that may be made during a multi-stage ultrasonic operation. As shown, the illustrative tissue parameters are impedance magnitude, measured in ohms, and impedance phase, measured in degrees. As described above, other parameters are possible, including rate of change of impedance. The illustrative impedance measurements illustrate the stages of the multi-stage operation. Stage 1002 corresponds to before the operation, when no tissue is in the jaw of the end effector. Measured impedance may correspond to an “open circuit” amount (i.e., effectively infinite). Stage 1004 corresponds to grasping tissue between the jaws, and may include pre-heating. Stage 1006 corresponds to activation of therapeutic ultrasound energy, including sealing and transection. As shown, during stage 1006 impedance magnitude initially drops and then gradually increases. The phase 1006 may be completed when the measured impedance magnitude reaches a predetermined threshold. Phase 1008 corresponds to no tissue in the jaw of the end effector, indicating that transection is complete and the jaw has opened.

Accordingly, the control element may cause the vessel sealing stage to be delivered until the tissue reaches a specified threshold value for impedance, rate of change of impedance, or other determined tissue value, as opposed to typical systems that may deliver energy for a fixed duration of time. As another example, the control element may deliver multiple power levels for the vessel sealing stage based on impedance of the tissue in the jaws. Accordingly, the disclosed technologies may provide optimized energy delivery for vessels of different sizes.

Further, by utilizing information about the tissue/vessel with an RF sensing signal as described in connection with FIGS. 9-10, advanced ultrasound operation is improved such that the changes from the different states (e.g., preheating, vessel sealing, transection) are not time-based but instead based on real-time information about the tissue. Accordingly, instead of completing the vessel sealing time in 8 seconds (or another predetermined duration) for every vessel, the vessel sealing time will be completed when a desired tissue characteristic is achieved, such as tissue impedance, the rate of impedance change, the amount of current able to go into the tissue, etc. Accordingly, compared to existing approaches, the technique disclosed in connection with FIG. 9 may improve sealing and transection performance, for example by reducing total time for small vessels, increasing time to ensure complete sealing for larger vessels, or otherwise optimizing vessel sealing based on real time sensing.

Referring now to FIG. 11, a method 1100 for controlling an energy-based surgical instrument 102 is shown. The method 1100 may be executed by the controller 702, the generator 106, and/or one or more other microcontrollers or other control elements of the system 100. The method 1100 begins in block 1102, in which the control element measures one or more mechanical parameters of the device jaws (e.g., the blade 130, the jaw clamp 132, and/or additional features of the end effector 120). In some embodiments, in block 1104 the control element measures an angle of initial tissue contact. In some embodiments, in block 1106 the control element measures timing of the initial tissue contact. In some embodiments, in block 1108 the control element measures an angle of jaw opening. In some embodiments, in block 1110 the control element measures force on the jaws to close. For example, in an embodiment with motorized jaws, the control element may measure motor torque, motor current, or otherwise monitor operation of the motorized jaws to determine the force of jaw closure.

In block 1112, the control element senses one or more tissue parameters using RF energy or ultrasonic energy. The control element may perform measurements using subtherapeutic and/or therapeutic energy levels. In some embodiments, in block 1114 the control element measures tissue impedance. In some embodiments, in block 1116 the control element measures ultrasonic transducer impedance. In some embodiments, in block 1118 the control element measures impedance over time (including change in impedance, rate of change of impedance, or other measures of impedance over time). In some embodiments, in block 1120 the control element measures impedance spectroscopy of the sensing signal, which may used to determine tissue composition. For example, the control element may measure tissue impedance at multiple frequencies to determine the tissue composition. Of course, in other embodiments other tissue parameters may be measured for the tissue within the jaws of the device 102.

In block 1122, the control element determines one or more energy delivery parameters based on a combination of the measured mechanical parameter(s) and the sensed tissue parameter(s). For example, the control element may determined whether to start, stop, or adjust energy delivery based on the measured mechanical parameters in combination with the measured tissue parameters. The combination of mechanical parameters and tissue parameters may be used to intuit between differing aspects of the device's operation order to providing situation awareness to the generator, smart device, or hub of what job or portion of the actuation is in process. This enables control of the jaws to be adapted in their portion in real-time, enabling switching between energy modalities, automatically opening or finishing clamping, or adjusting clamp arm/jaw pressure based on the circumstances.

In some embodiments, in block 1124 the control element determines the type of tissue clamped between the jaw based on the mechanical parameter and the tissue parameter. The energy delivery parameter may be adjusted based on the tissue type. In some embodiments, in block 1126 the control element determines the current operation stage or phase (e.g., pre-heating, vessel sealing, or transection) based on the mechanical parameter and the tissue parameter. In some embodiments, in block 1128, the control element selects an energy modality (e.g., RF versus ultrasound) based on the mechanical parameter and the tissue parameter. In block 1130, the control element may determines timing for a change in energy modality (e.g., when to start and/or stop one or more of the energy modalities). In some embodiments, in block 1132 the control element determines a power level or amplitude of the energy modality.

After determining the energy delivery parameter, the method 1100 is completed. The control element may cause the surgical instrument 102 to apply a selected energy modality (e.g., RF and/or ultrasound) using the determined energy delivery parameter. The method 1100 may be subsequently and/or repeatedly executed during the energy-based operation in order to further control energy delivery.

For example, the time of first closure experienced load is a good proxy for the thickness of the tissue, and can additionally be used in combination with force on the jaws or time to tissue creep stabilization as a good proxy for tissue compressibility.

As another example, angle of jaw can be used to in combination with energy activation state to determine if the user is interacting with a solid organ or other larger structure where the feathering technique is used to both debunk the tissue while its being clamped rather than conventional tissue welding which is used in the fully clamped state only.

As another example, force in the jaws or force/torque/current on the actuation motor can be used for local pressure, location of thickness within the jaws, compressibility/creep of the tissue, tissue debunking magnitude in order to determine the type, density, creep aspects, or response to the energy applied.

As another example, tissue impedance may be used a means to determine the water content and/or extent of the tissue welding done by the power applied. Tissue impedance over a range of frequencies (frequency spectroscopy) can be used to identify the tissue or properties of the tissue due to each tissue varying response to differing frequencies. Transducer impedance of an ultrasonic device 102 could be used as a measure of the force applied through the tissue to the blade 130 and its ability to maintain agitation.

While the disclosure has been illustrated and described in detail in the drawings and foregoing description, such an illustration and description is to be considered as illustrative and not restrictive in character, it being understood that only illustrative embodiments have been shown and described and that all changes and modifications that come within the spirit of the disclosure are desired to be protected.

There are a plurality of advantages of the present disclosure arising from the various features of the methods, apparatuses, and systems described herein. It will be noted that alternative embodiments of the methods, apparatuses, and systems of the present disclosure may not include all of the features described yet still benefit from at least some of the advantages of such features. Those of ordinary skill in the art may readily devise their own implementations of the methods, apparatuses, and systems that incorporate one or more of the features of the present invention and fall within the spirit and scope of the present disclosure as defined by the appended claims.

1. A method for controlling a surgical instrument, the method comprising:
applying, by a control element, a subtherapeutic radio frequency (RF) signal to tissue of a patient with an electrode of an end effector of the surgical instrument;
activating, by the control element, a first stage of an ultrasonic operation, wherein the first stage applies ultrasonic energy to an ultrasonic blade of the end effector of the surgical instrument;
measuring, by the control element, a tissue parameter with the subtherapeutic RF signal while activating the first stage of the ultrasonic operation;
determining, by the control element, whether the tissue parameter has a predetermined relationship to a predetermined threshold; and
activating, by the control element, a second stage of the ultrasonic operation in response to determining that the tissue parameter has the predetermined relationship to the predetermined threshold.

2. The method of claim 1, wherein the tissue parameter comprises tissue impedance.

3. The method of claim 1, wherein the tissue parameter comprises change in tissue impedance.

4. The method of claim 1, wherein the first stage comprises a pre-heating stage and the second stage comprises a vessel sealing stage.

5. The method of claim 4, wherein activating the second stage of the ultrasonic operation comprises applying ultrasonic energy to the ultrasonic blade at an energy level based on the tissue parameter.

6. The method of claim 1, further comprising:
measuring, by the control element, the tissue parameter with the subtherapeutic RF signal while activating the second stage of the ultrasonic operation;
determining, by the control element, whether the tissue parameter has a predetermined relationship to a second predetermined threshold; and
activating, by the control element, a third stage of the ultrasonic operation in response to determining that the tissue parameter has the predetermined relationship to the second predetermined threshold.

7. The method of claim 6, wherein the first stage comprises a pre-heating stage, the second stage comprises a vessel sealing stage, and the third stage comprises a transection stage.

8. The method of claim 7, wherein:
activating the first stage of the ultrasonic operation comprises applying ultrasonic energy at a first energy level;
activating the second stage of the ultrasonic operation comprises applying ultrasonic energy at a second energy level lower than the first energy level; and
activating the third stage of the ultrasonic operation comprises applying ultrasonic energy at a third energy level higher than the second energy level.

9. The method of claim 6, further comprising:
measuring, by the control element, the tissue parameter with the subtherapeutic RF signal while activating the third stage of the ultrasonic operation;
determining, by the control element, whether the tissue parameter has a predetermined relationship to a third predetermined threshold; and
deactivating, by the control element, the ultrasonic operation in response to determining that the tissue parameter has the predetermined relationship to the third predetermined threshold.

10. A method for controlling a surgical instrument, the method comprising:
measuring, by a control element, a first mechanical parameter of an end effector of the surgical instrument;
sensing, by the control element, a first tissue parameter while applying energy to tissue of the patient, wherein the energy comprises radio frequency (RF) energy or ultrasonic energy; and
adjusting, by the control element, delivery of therapeutic energy with the surgical energy based on the first mechanical parameter and the first tissue parameter.

11. The method of claim 10, wherein the first mechanical parameter comprises angle of initial tissue contact or time of initial tissue contact.

12. The method of claim 10, wherein the first mechanical parameter comprises force on the jaws.

13. The method of claim 10, wherein the first tissue parameter comprises tissue impedance.

14. The method of claim 10, wherein the first tissue parameter comprises transducer impedance.

15. The method of claim 10, wherein the first tissue parameter comprises impedance over time.

16. The method of claim 10, wherein adjusting the delivery of the therapeutic energy comprises selecting an energy modality of the therapeutic energy, wherein the energy modality is selected from RF energy or ultrasonic energy.

17. The method of claim 16, wherein adjusting the delivery of the therapeutic energy comprises changing the energy modality of the therapeutic energy.

18. The method of claim 17, wherein adjusting the delivery of the therapeutic energy comprises determining a time for changing the energy modality.

19. The method of claim 10, wherein adjusting the delivery of the therapeutic energy comprises determining a power level of the therapeutic energy.

20. The method of claim 10, wherein adjusting the delivery of the therapeutic energy comprises starting or stopping the delivery of the therapeutic energy.
